(19) United States
(12) Patent Application Publication
(10) Pub. No.: US 2009/0076449 A1
(43) Pub. Date: Mar. 19, 2009

(54) COATING AND METHOD FOR APPLYING A COATING TO A MEDICAL INSTRUMENT, AND MEDICAL INSTRUMENT

(76) Inventors: John S. Geis, Bad Zwischenhahn (DE); Michael Braun, Backnang (DE); Adalbert Kovacs, Dossenheim (DE); Gerhard K. Wolf, Heidelberg (DE)

Correspondence Address: Mark J. Pandiscio;Pandiscio & Pandiscio, P.C., 470 Totten Pond Road, Waltham, MA, 02451-1914, US

(21) Appl. No.: 11/884,666

(22) PCT Filed: Feb. 15, 2006
(86) PCT No.: PCT/DE06/00281

§ 371 Date: Sep. 15, 2008

(30) Foreign Application Priority Data
Feb. 18, 2005 (DE) 10 2005 007 596.7

(51) Int. Cl. A61M 29/00; A61M 25/00

(52) U.S. Cl. 604/103.05

(57) ABSTRACT

The invention relates to a cover (1) for a medical instrument (3), which is characterized in that the cover (1) is a generally pipe-shaped body and consists of one single tape (11) which extends generally helicoidally about the axis of the pipe-shaped body. The invention furthermore relates to a process for applying a cover (1) onto a medical instrument (3) as well as to a medical instrument (3) which is provided with a cover (1).

The present invention relates to a cover and to a method for applying a cover to a medical instrument as well as to a medical instrument which is provided with a cover.

It is known to use covers with medical instruments. Depending on the field of application, these covers have different purposes and in particular can serve for protection of the medical instrument from contamination, for better insertability of the instrument and/or for other purposes, such as the transport of medicaments or other substances to predetermined locations. In the prior art, the latter-mentioned covers are generally applied onto a medical instrument in form of coatings, wherein the instrument is immersed into the material which is supposed to form the coating and the coating is thus created. Furthermore, it is known to apply a coating onto a medical instrument by spraying methods.

The disadvantage of these production methods for covers on a medical instrument are in particular that the production of a uniform thickness of the coating can only be created with considerable effort, as a high level of precision has to be complied with during the production process. In addition, even if it might be possible to produce a coating with generally uniform thickness with an increased level of precision, a uniform structure across the thickness and area of the coating can, however, not be provided. Such a structure is, however, of crucial importance, especially for the usage of the cover for the transport and delivery of substances in a vessel, such as a blood vessel.

The problem underlying the present invention is therefore to provide a cover for a medical instrument which does not have these disadvantages. Additionally, a process for applying the cover to the medical instrument should be provided which can easily be carried out. Finally, a medical instrument having a cover applied thereto should be provided, wherein in the applied state the cover should possess a high level of homogeneity and a reliable connection to the medical instrument.

The invention is based on the finding that this problem can be solved by producing the cover separately from the medical instrument. In addition the cover can be applied to the medical instrument in an appropriate way.

The problem is thus solved by a cover for a medical instrument, in particular an elongated medical instrument, wherein the cover is characterized in that it is a generally pipe-shaped body und consists of one single tape which extends generally helicoidally about the axis of the pipe-shaped body. The medical instrument to which the cover according to the invention is to be applied, according to the invention, is a medical instrument which only is temporarily inserted into the human or animal body. In contrast to implants or supports, such as stents, it is thus a removable medical instrument, where different requirements apply. The cover according to the invention is adapted for these medical instruments and can advantageously be used for these medical instruments, which will be explained in greater detail below with reference to the medical instrument and to the process for applying the cover.

The helicoidal extension of the tape about the axis of the pipe-shaped body is adapted, according to the invention, in such a way that tape extends at an angle of greater or smaller than 90° to the axis of the pipe-shaped body. Thereby a body can be provided which is longer then the width of the tape which is used.

Due to the pipe-shape of the cover, it will become possible to apply the cover easily to a medical instrument, such as for example a wire or a catheter. Furthermore, due to the assembly of the cover by a tape, which helicoidally extends, a certain level of stability of the cover can be assured. This is because the cover consists of one single part, i.e. one single tape. A coming apart or a detaching of different parts of the cover can thus be avoided. In addition, the wall thickness of the cover with the inventive design which only consists of the layer formed by the tape can be kept very small. Essentially, the wall thickness of the cover can namely correspond to the thickness of the tape from which it is made. According to the invention, preferably a tape is being used, which has a rectangular cross-section, i.e. which has a uniform thickness over the width of the tape. With such geometry of the tape homogeneity of the properties over its width can be assured and the process of producing the tape can be facilitated compared to tapes with varying thicknesses.

In a preferred embodiment, the edges of the tape which is helicoidally extending at least partially overlap. This embodiment is advantageous in that the production of the cover can be facilitated. Compared to a cover wherein the edges of the tape merely abut against each other and via this line of contact might be connected, the requirements towards the precision are lowered with an overlap of the edges. In addition, the hold of the individual helicoidal coils to each other can be improved. If the edges overlap, the individual helical coils do not only come in contact with each other along a line but via a contact area via which they can also be connected to each other.

Preferably, the cover according to the invention has a uniform wall thickness. According to the invention this can be realized by an overlap of the edges over half of the respective tape width. In this case the wall thickness of the cover is twice as large as the wall thickness of the tape. Alternatively, the uniform wall thickness may be achieved by positioning the helicoidal coils adjacent to each other such that they are only in contact with each other on the edges. It is, however, also possible to achieve the uniform, in particular even, wall thickness, by applying a certain pressure onto the overlap area of the tape. This can occur during the laying of the tape in its helicoidal shape or the pressure can be applied onto the cover after the shaping of the spiral.

Preferably, the cover consists of a porous material and has a homogeneous distribution of pore sizes. This type of cover is in particular advantageous for using the cover as a carrier and delivery means for substances. Herein, the pores may serve for receiving and transporting the substances. If these pores have a uniform size, an even filling of the pores with the substance and also an even release of the substance from the pores is achieved. The latter is of particular importance for usages of the cover, wherein the cover is supposed to supply medicaments or other substances, such as oxygen, to a location in a body.

The inventive cover can be designed such that the diameter of the cover, in particular the inner diameter of the cover decreases towards one end of the cover. This shape of the cover has several advantages. Firstly, a threading a medical instrument into the cover can be facilitated. The medical instrument can be inserted into the cover from the side having a larger diameter. Furthermore, the narrowed diameter of the cover can fixate the cover to the medical instrument. Furthermore, the shape of the cover is advantageous for medical instruments which have a diameter which changes, in particular increases, over its length. Such an instrument is for example a balloon catheter. In this case, the shape of the cover corresponds to the shape of the medical instrument and in particular during insertion of the balloon catheter into a vessel, a dislocation of the cover over and beyond the balloon can be avoided. Finally, the shape of the cover with different diameters can be advantageous during its production. The cover may be formed on a mandrel and due to the decreased diameter on one side may be easily removed from the mandrel.

Preferably, the cover consists of polytetrafluoroethylene and further preferably of expanded polytetrafluoroethylene (EPTFE). This material is advantageous both in view of its elasticity as well as in view of its tolerance. In particular due to the elasticity of this material it is suitable for a cover, which according to the present invention is applied onto a medical instrument and potentially may be subjected to an expansion in the applied state. During such an expansion, the pore size of the cover can be increased and its wall thickness can be decreased, without the risk of the bursting of the cover. In addition, EPTFE offers a porosity which can serve for receiving and targeted release of substances. The substances which are to be released from the cover can be received in the pores of the porous material and can be held there until they are to be released. Also other materials known from medical engineering can be used for the cover according to the invention, wherein the choice of material is made dependent of the intended utilization of the cover.

Preferably, the cover according to the invention serves as a release medium, in particular as a release medium for solutions and/or emulsions. Examples of such solutions and/or emulsions are in particular oxygen carriers, such as perfluorocarbon solutions. The cover of the invention can also serve as a release medium for medicaments, wherein also the medicaments can be introduced into the cover as solutions or emulsions, in particular as solutions or emulsions with low surface tension. For example medicament/alcohol solutions or emulsions can be used. A cover made of porous material is advantageous for these usages.

The cover for a medical instrument according to the invention, in particular for a medical instrument which is only temporarily inserted into a human or animal body, can be manufactured by a process, which is characterized in that a tape is coiled onto a mandrel, wherein the tape is arranged essentially helicoidally and on the edges at least abuts, in that the thus formed helicoidal arrangement is connected in a connection step and is removed from the mandrel after completion.

By using a tape which is brought into a helicoidal shape, the properties of the cover to be manufactured can easily be influenced. On the one hand, the stability of the cover in its longitudinal direction can be influenced by the selection of the angle at which the tape is coiled. In the other hand, the stability can be influenced by the selection of the contact area between adjacent helicoidal coils. Finally, by using a porous tape, homogeneity of the pores across the area of the cover can be achieved.

The connection step which is carried out according to the present invention may be a heating step, in particular a sintering step. Thereby the material of the tape can fuse at the contact points of adjacent tape sections. This kind of connection is advantageous, as a targeted connecting at the contact points is not necessary and this step is thus facilitated. The temperature of the connection step and its duration are selected in dependency of the material of the tape. In particular, the parameters are adjusted such that a certain degree of fusing occurs at the contact points, the material of the tape, however, does not melt completely. In particular with porous tape material such melting would result in a change of porosity and may even result in the loss of porosity.

The cover according to the invention can be manufactured according to the described process. In particular, the cover will be manufactures in such a way that after coiling the tape and a potentially connecting step, wherein the tape fuses, the cover will have a diameter which corresponds to the outer diameter of the medical instrument onto which the cover is to be applied. The outer diameter of an expandable medical instrument herein corresponds to the outer diameter in the relaxed that means unexpanded, state.

According to a further aspect of the invention, the invention relates to a process for applying a cover to a medical instrument. This process is characterized in that a prefabricated cover is slipped onto a medical instrument having an elongated body and the cover is being fixated, wherein the cover is only fixated over part of its length on the medical instrument.

By using a prefabricated cover, properties of the cover can be adjusted easily compared to a coating, wherein the layer is created on the instrument. In the inventive process, in a first step the cover is only fixated in the desired position. An attachment of the cover may then occur in a subsequent step.

The fixation of the cover can be realized by using an adhesive. Herein, the adhesive may be provided over the entire length of the cover between the cover and the medical instrument. It is, however, also possible to only provide adhesive in the area of the ends of the cover between the cover and the medical instrument. The advantage of a fixation step which is carried out before any attachment step that may be carried out is that the medical instrument can already be transported after the fixation without bearing the risk of the cover slipping off of the instrument. Dependent on the medical instrument, it may also be put to use with a cover which is only fixated at the instrument, which has, however, not been subject to any further attachment step. This application of the cover may for example be suitable for a wire as a medical instrument.

As the cover according to the present invention is only fixated over part of its length to the medical instrument, firstly the application of the cover can be facilitated. Furthermore, due to the partial fixation an area of the medical instrument can specifically be omitted. The omitted area may for example be the balloon area of a balloon catheter. A fixation in this area by means of adhesives may result in damaging the balloon during the expansion of the balloon or an expansion, that means the unfolding of a balloon which is generally folded, may be precluded. By a fixation exclusively outside of the expandable areas nevertheless a sufficient hold of the cover on the medical instrument can be assured which allows for an insertion of the instrument into a body vessel, such as a blood vessel, without risking a detaching of the cover.

According to the present invention an attachment step can be carried out after the fixation step. With an expandable medical instrument, such as for example a balloon catheter, the attachment step may be carried out on-site, for example by the surgeon.

The attachment step therefore according to one embodiment comprises a change of sectional area of the medical instrument. By means of such a change of sectional area, in particular increase of sectional area, a press fit connection between the medical instrument and the cover can be created. In addition to the mere force-based connection, friction occurs between the medical instrument and the cover during the increase of sectional area of the medical instrument and the thus effected increase of sectional area of the cover. At the contact area between the medical instrument and the cover therefore adhesion by friction occurs which also remains after a subsequent decrease of sectional area.

A change of sectional area may be effected for example with a balloon catheter by dilation of the balloon. This can be carried out during the utilization of the instrument. During its first usage, in particular if the cover is porous, the cover will thus be in its original state, in particular not in an over expanded state. In this state the receiving ability of the cover for substances in the pores can be maximal. It is, however, also possible to carry out a change of sectional area before distribution, that means at the production site of the medical instrument which is provided with the cover. Due to the increase of sectional area of the medical instrument and of the cover, the wall thickness of the cover is decreased compared to the wall thickness with which the cover had been produced from the tape. Also the pore size within a porous cover changes and increases with an increase of sectional area. For cases where the cover serves for delivery of substances, the release conditions are thereby improved.

Alternatively or additionally, the attachment step may also comprise a heating step. Herein it is possible to heat the medical instrument with the cover which is fixated thereon. Within the scope of the invention it is, however, also possible to exclusively heat the medical instrument. This kind of attachment is particularly suitable for medical instruments made of heat-conducting material, such as wires. With these instruments an exclusive heating of the instrument can be carried out by which the cover at its contact area to the instrument potentially slightly melts and thus becomes attached to the instrument. An influence on the properties, in particular on the side which is facing away from the medical instrument, does not occur herein.

According to a last aspect, the invention relates to a medical instrument having a cover provided over at least a part of its surface. The medical instrument is in particular characterized in that the cover is only fixated over part of its length onto the medical instrument and in that the cover is a generally pipe-shaped body, wherein the walls of the pipe-shaped body are formed by a helicoidally extending tape, wherein the edges of the tape at least abut against each other. The advantage of such a medical instrument is that the cover due to its design has certain stability in its longitudinal direction. In contrast to a cover where annular parts are connected to each other, a peeling of the cover from the instrument during insertion of the instrument into a vessel, such as for example a blood vessel, can not occur with the inventive medical instrument.

Furthermore, a possibility of combining different application kinds on the medical instrument is provided by the partial fixation of the cover on the medical instrument. Thereby, the properties of the medical instrument and its way of utilization can be accounted for. For example, adhering the cover to the instrument can be combined with an adhesion by friction. The cover is thus also suitable for instruments which have different properties in different areas.

The medical instrument is preferably a catheter, in particular a balloon catheter or a wire.

The cover of the medical instrument preferably is a cover according to the invention and/or is applied according to the application process of the invention onto the medical instrument.

Advantages and features which are described with reference to the cover of the invention, also are valid—where applicable—for the inventive application process as well as for the inventive medical instrument, respectively, and vice versa.

The invention will hereinafter be described with reference to the attached drawings, which relate to possible embodiments of the invention, wherein:

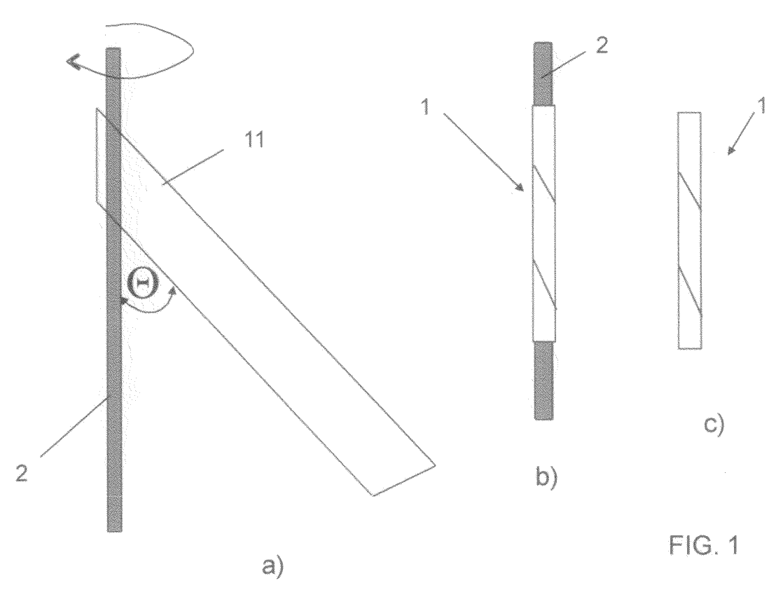

FIGS. 1a to 1c: show a schematic representation of the operating sequence of an embodiment of the production process;

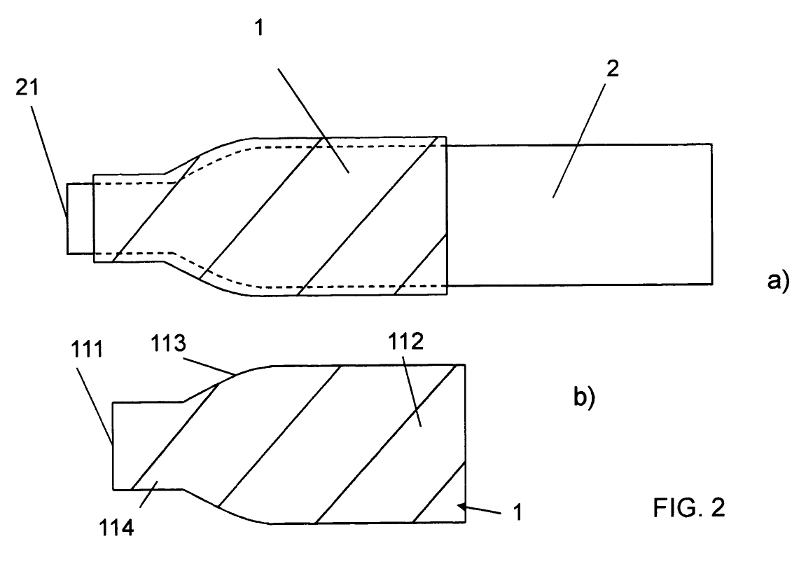

FIGS. 2a and 2b: show schematic representations of an embodiment of a cover according to the invention;

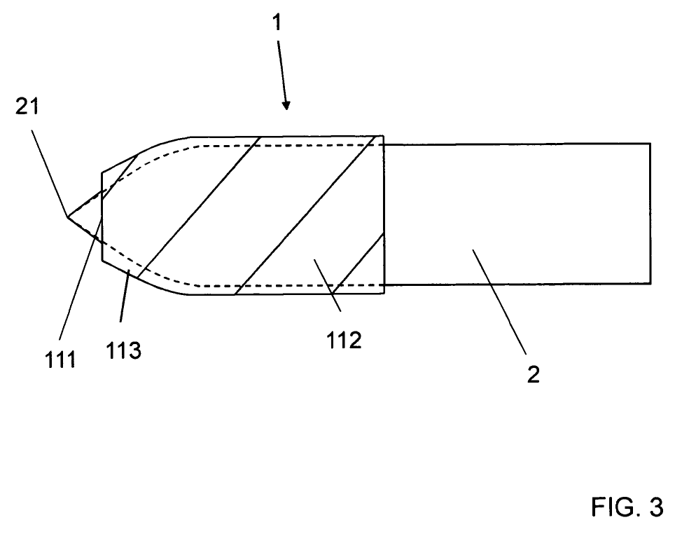

FIG. 3: shows a schematic representation of a further embodiment of a cover according to the invention; and

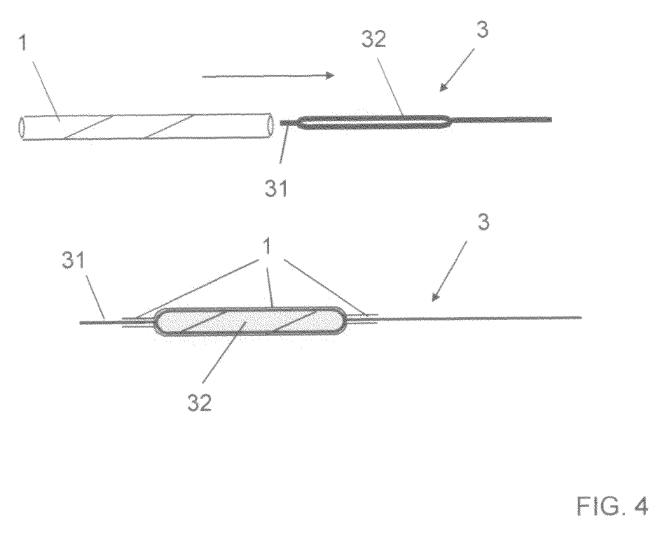

FIG. 4: shows a schematic representation of the operating sequence of an embodiment of the application process according to the invention.

The representations are not to scale.

In FIG. 1 a schematic depiction of an operating sequence of an embodiment of the production process of a cover according to the invention is shown.

In a first step a tape 11 made of elastic, preferably porous material is joined to a mandrel 2, which in the depicted embodiment can be a rod or a pipe. The tape 11 is joined at an angle θ to the axis of the longitudinally extending mandrel 2. Subsequently, the tape 11 is coiled onto the mandrel 2. This can be done, as shown in FIG. 1, by rotation of the mandrel 2 about its longitudinal axis. Due to the angle which is present between the axis of the mandrel 2 and the longitudinal direction of the tape 11, a helicoidal coil is created on the mandrel 2. This coiled state of the tape 11 on the mandrel 2 is shown in FIG. 1b.

In this state, the cover 1 can be subjected to a heating step, in particular a heat treatment. With an EPTFE tape for example a heating of the cover at 380° for 60 seconds is carried out. At this temperature the edges of the tape 11, which abut or even overlap, can fuse together. A complete melting of the cover 1 is, however, precluded due to the selected temperature and time period. The heating step is preferably carried out in a furnace, wherein the mandrel 2 together with the tape coiled thereon is inserted.

The cover 1 is cooled after the heating step. This is preferably achieved by mere exposure to the ambient temperature.

The cover 1 can be removed from the mandrel 2 after cooling down. In order to achieve this, the cover 1 can be moved relative to the mandrel 2 or the cover 1 is held and the mandrel 2 is moved relative thereto and thus pulled out of the cover 1.

After the production, hence, a cover 1 as shown in FIG. 1c is present. This cover 1 can be used for different purposes, in particular, the cover 1 may be applied on various medical instruments, such as for example balloon catheters or wires, such as guide wires. The medical instruments are herein realized for a temporary insertion into vessels, in particular into blood vessels. The application of the cover 1 is carried out in the state of the cover 1 that it has after the described production. The cover 1 will in particular not be compressed over its diameter before being applied onto the medical instrument.

An alternative to the shape of the cover 1 shown in FIG. 1 is depicted in FIG. 2. In this embodiment a mandrel 2 is used, wherein one end 21 has a decreased diameter compared to the further length of the mandrel 2. Thereby, the tape 11 coiled thereon will be brought into a funnel shape, wherein the transition between different diameters is preferably continuous in order to avoid steps at the cover 1. In the depicted embodiment the cover 1 has a cylindrical area 112 with a larger diameter, an adjacent conical transition area 113 and a cylindrical area 114 of smaller diameter. This area 114 is the end of the cover 1.

The advantage of this embodiment is in particular, that the shape of the cover 1 is a representation of the shape of the medical instrument 3 in the area where the cover 1 is to be applied. In this embodiment, the outlines of the cover 1 and of the medical instrument 3, as for example the distal end 31 and balloon area 32 of a balloon catheter 3, correspond. In the balloon area 32 the outline of the cover 1 corresponds during the application onto the catheter 3 to the outline of the balloon in its folded state. Furthermore, due to its shape, the cover 1 can be more easily removed, that means pulled of, from the mandrel 2. The further process steps, in particular the heating and the cooling of the cover 1 correspond to the process described with reference to FIG. 1.

In FIG. 3 a further embodiment of the cover 1 according to the invention is shown. In this embodiment, the mandrel 2 which is used has a tip 21. The tape 11 is coiled along the tip 21 and along the adjacent cylindrical part of the mandrel 2. Thereby, the cover 1 obtains a shape wherein a taper cone is adjacent to the cylindrical part 112. The shape does not transition into a further cylindrical part. In this embodiment, the tape 11 is joined to the mandrel 2 such that it is at a distance to the tip 21 of the mandrel 2. Thereby, also in this embodiment it is assured that a passage remains within the cover 1 through which a medical instrument 3 can be guided.

The embodiments of the cover 1 shown in FIGS. 1 to 3 can be applied to a medical instrument 3 as shown in FIG. 4. In the depicted embodiment, the medical instrument 3 is a balloon catheter. The cover 1, as can be derived from FIG. 4a, is slid onto the end of the balloon catheter 3, where the balloon 32 is provided. The cover 1 is pushed onto the catheter 3 until the cover 1 covers the balloon area 32 of the catheter 3. The cover 1 can be fixated in this position. This can for example be done by previous or subsequent application of an adhesive to the side of the balloon 32 which faces away from the end 31 of the balloon catheter 3 over which the cover 1 has been slid on. In addition, adhesive may be provided at the front end 31 of the balloon catheter 3.

After the cover 1 has been brought into this position and has been fixated, the balloon 32 can be pressurized, that means dilated. Due to the increase in diameter of the balloon 32 a press fit of the cover 1 on the balloon 32 is achieved. If the balloon 32 is subsequently collapsed again, the connection between the balloon material and the cover 1 will be maintained. The maintaining of the connection is based on the fact that in addition to a mere force-based connection between the balloon material and the cover 1 also an adhesion due to the friction occurring during the dilation of the balloon occurs between the components.

The invention is not limited to the depicted embodiments.

The cover preferably consists of one single layer, which is formed by a tape. Thereby the overall thickness of the wall of the cover can be minimized. Furthermore, homogeneity within the cover can be provided and the production of the cover can be carried out in an easy and rapid fashion.

In addition, the cover according to the invention is designed such that it can be completely penetrated by a medical instrument. For this purpose the cover has two opposing openings, which form the ends of the pipe-shaped cover. Thereby, it is possible to bring the cover onto an elongated medical instrument to a targeted position where it is supposed to be effective. With the cover according to the invention, this position does not necessarily have to be the end of the medical instrument.

1. Cover for a medical instrument (3), the cover comprising (1) a generally pipe-shaped body comprising one single tape (11) which extends generally helicoidally about the axis of the pipe-shaped body.

2. Cover according to claim 1, wherein the edges of the tape (11) which extends helicoidally at least partially overlap.

3. Cover according to claim 1, wherein the cover (1) has a uniform wall thickness.

4. Cover according to claim 1, wherein the cover comprises a porous material and has a homogeneous distribution of pore size.

5. Cover according to claim 1, wherein the diameter of the cover (1), in particular the inner diameter of the cover, decreases towards one end (111) of the cover (1).

6. Cover according to claim 1, wherein the cover (1) consists of PTFE.

7. Cover according to claim 1, wherein the cover serves as a release medium.

8. Process for applying a cover onto a medical instrument (3), the process comprising slipping a prefabricated cover (1) onto a medical instrument (3) having an elongated body; and fixating the cover, wherein the cover (1) is only fixated over part of its length to the medical instrument (3).

9. Process according to claim 8, wherein the attachment step comprises a change of sectional area of the medical instrument (3).

10. Process according to claim 8, wherein the attachment step is carried out during the first usage of the medical instrument (3).

11. Medical instrument comprising a cover (1) provided over at least a part of its surface, wherein the cover (1) is only fixated over part of its length onto the medical instrument (3); and further wherein the cover (1) comprises a generally pipe-shaped body, wherein the walls of the pipe-shaped body are formed by a helicoidally extending tape (11), wherein the edges of the tape (11) at least abut against each other.

12. Medical instrument according to claim 11, wherein the medical instrument is designed for temporary insertion in a body vessel.

13. Medical instrument according to claim 11, wherein the medical instrument is a balloon catheter.

14. Medical instrument according to claim 13, wherein the cover (1) is exclusively fixated to non-expandable areas (31) of the medical instrument (3).

15. Medical instrument according to claim 11, wherein the medical instrument is produced by slipping a prefabricated cover (1) onto a medical instrument (3) having an elongated body; and fixating the cover, wherein the cover (1) is only fixated over part of its length to the medical instrument (3).

16. Cover according to claim 1, wherein the cover (1) consists of EPTFE.

17. Cover according to claim 1, wherein the cover serves as a release medium for solutions and/or emulsions.
